(19) United States
(12) Patent Application Publication
(10) Pub. No.: US 2026/0306745 A1
(43) Pub. Date: Oct. 8, 2026

(54) Medicament delivery device and medicament delivery assembly

(71) Applicant: SHL Medical AG, Zug (CH)

(72) Inventor: Justin Stewart, Deerfield Beach, FL (US)

(21) Appl. No.: 19/477,941

(22) PCT Filed: Apr. 12, 2024
(86) PCT No.: PCT/EP2024/059947

§ 371 Date: Oct. 23, 2025

Related U.S. Application Data
(60) Provisional application No. 63/462,296, filed on Apr. 27, 2023.

(30) Foreign Application Priority Data
Jul. 27, 2023 (EP) 23188186.3

(51) Int. Cl. A61M 5/24; A61M 5/20

(52) U.S. Cl. CPC A61M 5/2429; A61M 5/2033

(57) ABSTRACT

The present disclosure provides a medicament delivery device for accommodating a medicament container. The medicament delivery device comprises a base unit with a housing extending along a proximodistal axis and a drive unit arranged to be subject to an expelling movement relative to the base unit into a proximal direction, to act on the medicament container for expelling a medicament. The medicament delivery device further comprises a signal member extending longitudinally and being configured to interact with the drive unit and the base unit to be displaced towards distally upon completion of the expelling movement, and to thereby generate a confirmation signal. One of the signal member and of the drive unit comprises a feedback structure extending along the axial direction and comprising a plurality of indentations and/or protrusions The other one of the signal member and of the drive unit comprises an engaging structure for interacting with the feedback structure. The expelling movement causes the engaging structure to slide along the feedback structure and to thereby sequentially engage a plurality of the indentations and/or protrusions to cause a sequence of feedback signals.

### TECHNICAL FIELD

The invention is in the field of medicament delivery devices. More in particular, it relates to an automatic medicament delivery device providing audible, tactile and/or haptic feedback to a patient who uses it. It further relates to an assembly comprising such a device, pre-assembled with a medicament container.

### BACKGROUND

Medicament delivery devices for automatic delivery of a medicament by self-administration are well-known. Especially, they may be equipped to accommodate a medicament container, for example a medicament container with a septum to be perforated immediately prior to use, or a syringe. Often, the medicament delivery device and the medicament container are pre-assembled to constitute a medicament delivery assembly for self-administration.

Such medicament delivery devices and medicament delivery assemblies should generally be safe to use and easy to handle, and they should ensure delivery of the designated dose of the medicament. In view of this, it may be important that the patient receives a feedback signal during and/or after delivery of the medicament.

WO 2011/123024 discloses a medicament delivery device for a syringe as a medicament container, wherein the medicament delivery device provides an audible, tactile or visible confirmation to a patient when an injection has been made. To this end, the medicament delivery device comprises a signal generating member being an elongated U-bracket. The U-bracket is allowed to be displaced towards distally as soon as the plunger rod that displaces the plunger of the syringe is in the end position. The drive spring, which displaces the plunger rod during delivery, then acts to move the U-bracket towards distally until it taps against the inner surface of the housing, resulting in the desired confirmation signal.

U.S. Pat. No. 7,758,550 and WO 2017/129337 disclose medicament delivery devices in which a plunger rod moves, for delivering the medicament, urged by a biasing member, towards distally. This movement of the plunger rod towards distally causes latching elements or bumps to interact with an engaging structure moved relative thereto in an axial direction, so that discrete acoustic/audible ‘click’ signals are generated during medicament delivery.

Both, acoustic feedbacks during medicament delivery and an acoustic confirmation signal after completed medicament delivery have their advantages. The former indicates to the patient that the device is working and that delivery is going on. When the patient does not hear (or feel) the signal any more, she/he might, however, be uncertain whether delivery has been completed or not. The confirmation signal after completion deals with this, however, there is no feedback during delivery.

### SUMMARY

For the above reasons, it would be desirable to combine both, signal generation during delivery and signal generation thereafter. Accordingly, it is an object of the present invention to provide a medicament delivery device that overcomes at least some of the shortcomings of the prior art and that combines signal generation during delivery and signal generation thereafter in a single medicament delivery device. It is a further object to provide a medicament delivery device that does not require too many additional parts and structures compared to prior art devices and that is economical to manufacture and to assemble.

These and other objects are achieved by the invention as defined in the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which during use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which during use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component.

Similarly, the terms “transverse”, “transversal” and “transversally” refer to a direction generally perpendicular to the longitudinal direction.

When a component is said to move proximally, distally, axially in a proximal direction, axially in a distal direction, in a circumferential direction, or equivalent terms, the movement is relative to the housing of the injection device, unless mentioned otherwise.

According to an aspect of the present invention, a medicament delivery device for accommodating a medicament container is provided. The medicament delivery device comprises a base unit with a housing extending along a proximodistal axis and a drive unit arranged to be subject to an expelling movement relative to the base unit into a proximal direction, to act on the medicament container for expelling a medicament therefrom. If the medicament container has a plunger or stopper, the drive unit may for example act, by being moved into the proximal direction, on the plunger or stopper to expel the medicament via a needle, which needle may belong to the medicament delivery device, or, for example if the medicament container is a syringe, to the medicament container.

The medicament delivery device further comprises a signal member, the signal member extending longitudinally and being configured to interact with the drive unit and the base unit to be displaced towards distally upon completion of the expelling movement, and to thereby generate a confirmation signal once the expelling movement has been completed.

One of the signal member and of the drive unit comprises a feedback structure. The feedback structure extends along the axial direction and comprises a plurality—in particular a spatial sequence extending axially—of indentations and/or protrusions. The other one of the signal member and of the drive unit comprises an engaging structure for interacting with the feedback structure. The expelling movement causes the engaging structure to slide along the feedback structure and to thereby engage, one after the other, a plurality of the indentations and/or protrusions to cause a sequence of feedback signals.

Therefore, in the approach according to the present invention, the signal member causes both, a sequence of feedback signals during the expelling movement—thus, during the medicament delivery—and a confirmation signal thereafter. Without necessitating additional elements or other parts, it thereby combines the advantages of the two approaches of the prior art.

In embodiments, the signal member may have a distal base and a plurality of arms extending towards proximally therefrom. Especially, it may have an overall U-shape—a bracket shape—, with a transversal bridge portion arranged distally, as the distal base, and with two arms extending towards proximally therefrom. In this, the biasing member (for example drive spring), which biases the drive unit to be subject to the expelling movement when released, may act between the transversal bridge portion and the drive unit. When the expelling movement has been completed, the signal member is allowed to be moved towards distally relative to the base unit so that the biasing member pushes the signal member towards distally and causes it to hit a distal end wall of the base unit, thereby generating the confirmation signal.

In particular, the signal member and its working principle may be of the kind described in WO 2011/123024.

The drive unit may comprise a plunger rod equipped to act on a plunger or stopper of the medicament container for expelling the medicament therefrom. Especially, it may be the plunger rod that comprises the engaging structure (if the signal member has the feedback structure) or the feedback structure (if the signal member has the engaging structure).

The signal member in the initial state—prior to the expelling movement—may extend along at least a substantial portion of the plunger rod's axial extension. For example, if the signal member has the plurality of arms extending from the distal base, the plunger rod may be at least partially accommodated between the arms in the initial state. Thus, the arms extend along different sides of the plunger rod—for example along opposite sides if there are two arms.

In this, the protrusions of the feedback structure may be inwardly facing signal member protrusions. The engaging structure of the plunger rod, which together with the feedback structure causes the sequence of signals, may then be constituted by an outwardly facing signal member protrusion, or for example by a shoulder or a distal or proximal end thereof.

Alternatively, the plunger rod may comprise the feedback structure, for example in the form of a spatial sequence of protrusions and/or indentations along its axial extension. Then, the engaging structure may comprise at least one inwardly facing protrusion, for example near the proximal end of the arms of the signal member.

It is possible that both, the signal member and the drive unit (for example the plunger rod) comprises an axially extending spatial sequence of protrusions and/or indentations, whereby both structures simultaneously act as feedback structure and as engaging structure.

In embodiments, the base unit comprises a tubular portion with a cavity open towards proximally, wherein the signal member and the drive unit are, in the initial state prior to the expelling movement, at least partially accommodated in the cavity.

Such tubular portion may for example belong to a distal end cap member of the base unit, the distal end cap member having a distal end wall.

The signal member may in the initial position be such that there is a gap between the distal base—for example the transversal bridge—and the distal bottom of the cavity. The signal member may comprise outwardly protruding support portions—for example one for every arm—that secure the signal member from being pushed, by the biasing member—further into the cavity. For example, the support portions may be arranged immediately proximally of a mouth of the cavity, abutting against a proximal end of the tubular portion.

The confirmation signal may be initiated by the plunger rod moving out of the space between the arms of the signal member, i.e., the distal end portion of the plunger rod moving past the proximal end of the signal member. Especially, the arms may be biased towards radially-inwardly, pressing against the surface of the plunger rod. As soon as the plunger rod has moved out of the space between the arms of the signal member, the arms are allowed to flex radially-inwardly. As a result, the outwardly protruding support portions get out of engagement with the tubular portion's mouth, so that the signal member can be pushed, by the biasing member, which urges the drive unit towards proximally, against a resistance by the medicament container, towards distally, further into the cavity. This closes the mentioned gap, and the drive unit taps against the bottom of the cavity, and thereby generates the confirmation signal.

A distal end cap member of the described kind may form part of the housing of the medicament delivery device, for example together with a housing sleeve (thus, a tubular element of the housing). In addition to comprising a housing sleeve and/or a distal end cap member, the housing may comprise a container housing accommodated within the sleeve and providing a seat for the medicament container.

Initial activation of the drive unit by releasing it may, as is known from the prior art, be automatic. For example, the medicament delivery device may comprise a needle guard protecting a needle (of the medicament delivery device or of the medicament container, the needle being for injecting the expelled medicament under the patient's skin) after a proximal end cap or similar has been removed, immediately prior to use. The automatic release may be caused by the needle guard being displaced, against a biasing force, towards distally when the patient presses the medicament delivery assembly against the dose delivery site. To this end, the medicament delivery device may comprise a drive locking member that prevents the drive unit from moving towards proximally prior to release. Release may for example be due to a rotation of the drive locking member, such rotation caused by the displacement of the needle guard towards distally.

In addition to concerning a medicament delivery device, the present invention also concerns a medicament delivery assembly comprising the medicament delivery device of the kind described in the present text, pre-assembled with a medicament container, for example a medicament container with a septum to be pierced by a needle of the medicament delivery device prior to use, or a syringe with a pre-assembled needle.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

### BRIEF DESCRIPTION OF THE DRAWINGS

Embodiments of the present disclosure will now be described by way of example only and with reference to the following accompanying drawings.

The drawings show:

FIG. 1 a view of an embodiment of a medicament delivery assembly comprising the medicament delivery device and a medicament container;

FIG. 2 a housing sleeve of the medicament delivery device;

FIG. 3 a needle guard of the medicament delivery device;

FIG. 4 the medicament delivery assembly of FIG. 1, with a proximal end cap and a needle cap assembly removed, and without the housing sleeve and the needle guard, the medicament container and a container housing being shown transparent;

FIG. 5 a sub-assembly of the medicament delivery assembly, the sub-assembly comprising a plunger rod, a signal member and drive spring being a biasing member;

FIG. 6 the sub-assembly of FIG. 5 without the plunger rod;

FIG. 7 a distal end cap member of the medicament delivery assembly; and;

FIG. 8 the sub-assembly of FIG. 5 assembled with the distal end cap member, in longitudinal section.

### DETAILED DESCRIPTION

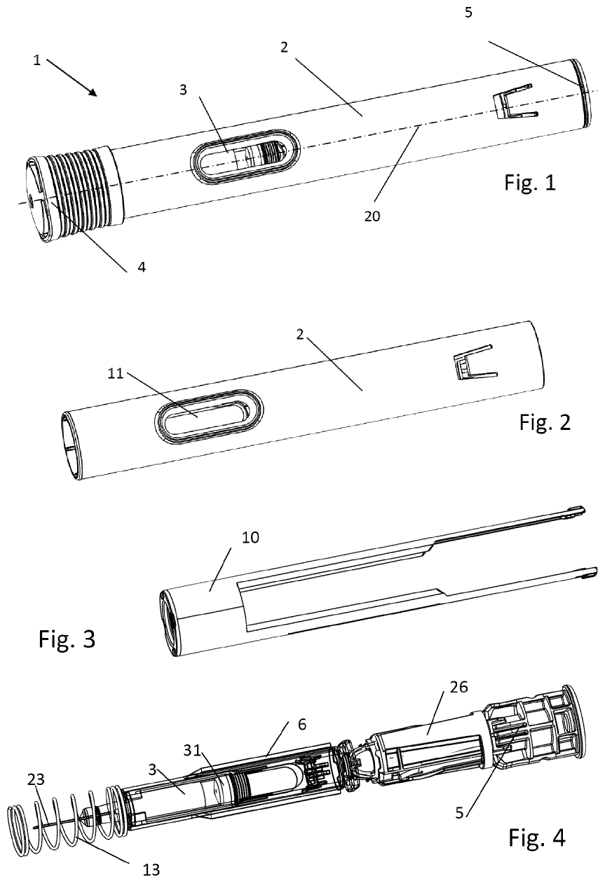

The medicament delivery assembly 1 shown in FIG. 1 comprises a medicament delivery device and a medicament container 3. The medicament delivery device comprises a base unit with a housing, the housing comprising a housing sleeve 2 and a distal end cap member 5. A proximal end cap 4 protects, possibly together with a needle cap assembly that comprises at least one needle cap, the needle of the medicament delivery assembly during transportation and (short-time) storage.

Prior to use, the proximal end cap 4 and the needle cap assembly are removed.

FIG. 2 shows the housing sleeve 2. The housing sleeve has a housing window 11 through which the medicament container 3 is visible for the patient to inspect the content thereof.

FIG. 3 depicts the so-called needle guard 10 (or ‘needle shield’ or ‘needle cover’). FIG. 4 shows the medicament delivery assembly of FIG. 1, with the proximal end cap and the needle cap assembly removed, and without the housing sleeve 2 and the needle guard 10.

The assembly of FIG. 4 together with the needle guard 10 and the housing sleeve 2 thus constitutes the medicament delivery assembly immediately prior to its use.

In the depicted embodiment, the base unit of the medicament delivery device comprises, in addition to the housing sleeve 2 and the distal end cap member 5, also a container housing 6 accommodating the medicament container. The container housing is for example transparent or has a window, so as to allow the patient to inspect the medicament through the housing window 11. The container housing 3 in the depicted embodiment is a syringe that comprises a needle 23. Alternatively, the medicament container could be a container comprising a septum to be pierced, prior to use, by a needle that belongs to the medicament delivery device.

The medicament delivery device further comprises a drive locking member 26 (sometimes called ‘rotator’) and a plunger rod, together with further elements described in more detail hereinafter. The plunger rod, if applicable together with further parts moving with it, forms the drive unit.

As is known in the field, after removal of the proximal end cap and the needle cap assembly, the needle guard 10 is forced, by a needle guard spring 13 into a proximal position in which it protects the needle 23 of the medicament delivery assembly.

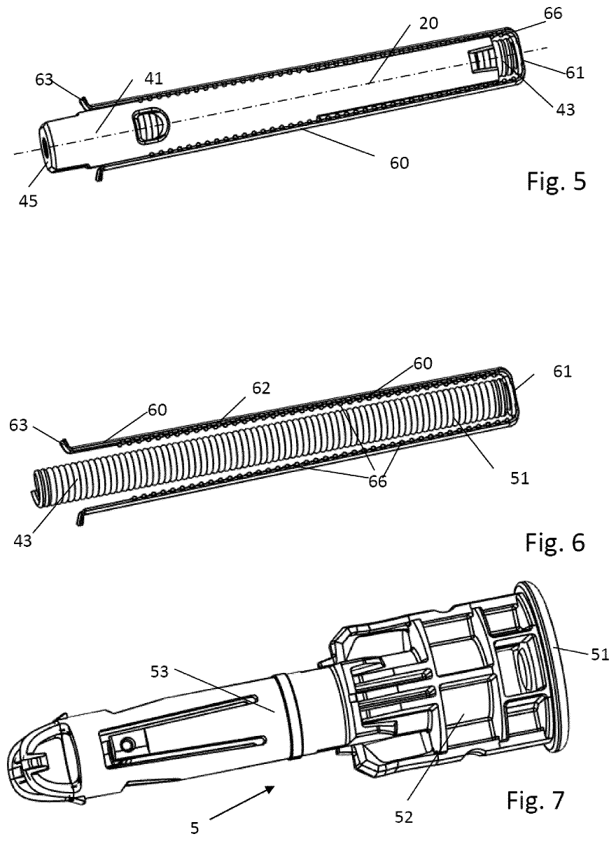

For activation, the patient presses the medicament delivery assembly of FIGS. 2-4, thus with the proximal end cap and, if applicable, the needle cap assembly, removed, against the dose delivery site and thereby displaces the needle guard 10 towards distally relative to the housing, against a spring force of the needle guard spring 13. As a result, the needle 23 becomes exposed and can penetrate the patient's skin at the dose delivery site. At the same time, the needle guard 10 interacts with the drive locking member 26 to rotate the same about the proximodistal axis 20 (see FIG. 1 and FIG. 5), by a certain angle. The drive locking member 26 will, by this movement, release the drive unit and allow it to move axially into the proximal direction (expelling movement) so that the plunger rod acts on the medicament container's stopper 31 (or plunger). Thereby, it activates the medicament delivery.

There are several possible mechanisms by which a movement of an activation member—such as the needle guard 10—relative to the housing can activate the medicament delivery. Since the present invention does not depend on the nature of this activation, it is not described in any more detail here.

FIGS. 5-8 illustrate how signals are generated during and after medicament delivery, initiated by the drive unit.

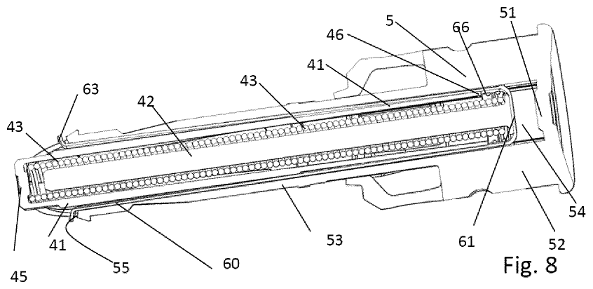

FIG. 5 shows the plunger rod 41 together with the biasing member—here being a drive spring 43—and the signal member 60. FIG. 6 depicts the elements of FIG. 5 without the plunger rod 41. FIG. 7 shows the distal end cap member 5, and FIG. 8 shows the elements of FIG. 5 and the distal end cap member as assembled together and in section.

The plunger rod 41 has the shape of a sleeve at least partially closed towards proximally by a proximal end bottom 45 that, during the expelling movement is pressed against the stopper 31 of the medicament container. The drive spring 43 extends around a shaft of a spring guide 42 into an interior of the plunger rod 41, with its proximal end abutting against the proximal end bottom 45.

The signal member 60 has the shape of a U-bracket, with a distal transversal bridge 61 and two arms 62 extending towards proximally therefrom. At the proximal end of the arms 62, there is a radially outwardly extending, flange like support portion 63. As can best be seen in FIG. 6, the signal member has, along its arms 62, a plurality of inwardly protruding signal member protrusions 66. The signal member protrusions 66 may be arranged at regular distances, or the distances between the signal member protrusions may differ.

The distal end cap member 5 has a head portion 52 with a distal end wall 51. A tubular portion 53 having a cavity 54 open towards proximally extends into the head portion 52, so that the cavity 54 is delimited, towards distally, by the distal end wall 51 (see FIG. 8).

As can best be seen in FIG. 8, the drive spring 43 in the initial configuration, prior to the onset of the expelling movement is in an energy storage state, i.e. it is tensioned by being compressed between the transversal bridge 61 of the signal member 60 and the proximal end bottom 45 of the plunger rod 41. The support portions 63 of the signal member 60 are proximally of the tubular portion 53 of the distal end cap member, whereby the proximal end face 55 of the tubular portion prevents the signal member 60 from being displaced, by the spring force of the drive spring 43, towards distally further into the cavity 54.

As soon as the drive locking member 26 (and/or other mechanism), due to an activation by the user, ceases to impede a movement of the plunger rod 41 towards proximally, the spring force of the drive spring 43 causes the expelling movement by driving the plunger rod 41 towards proximally, thereby acting on the stopper 31 of the syringe to expel the medicament via the needle 23. This movement towards proximally causes the plunger rod 41 to move out of the space between the arms 62 of the signal member 60. During this movement, the distal end portion 46 successively passes along the signal member protrusions 66. Every time the distal end portion 46 gets out of engagement with one of the signal member protrusions 66, a signal will be caused due to the signal member 60 and/or the plunger rod's distal end portion 46 giving in due to resiliency in it. The result will be the sequence of acoustic feedback signals during the expelling movement. It should be noted that the feedback signals may provide a tactile and/or haptic feedback as well to the user. Therefore, in this embodiment, the distal end portion 46 acts as the engaging structure for generating, together with the feedback structure constituted by the spatial sequence of signal member protrusions 66, the sequence of signals during the expelling movement.

The arms 62 of the signal member 60 are biased towards radially-inwardly, whereby they press against the outer surface of the plunger rod 41. As soon as the distal end portion 46 of the plunger rod 41 passes the distal end of the arms 62, the arms 62 are allowed to flex inwardly. Thereby, their support portions 63 get out of engagement with the proximal end face 55. The signal member 60 is thereby allowed to be moved towards distally by the spring force of the drive spring 43, whereby the transversal bridge taps against the distal end wall 51 of the distal end cap member 5. This tapping against the distal end wall 51 causes the confirmation signal after the expelling movement. The mechanism of generating a confirmation signal in this way is described in more detail in WO 2011/123024.

Various other embodiments may be envisaged.

For example, instead of the signal member 60 comprising the arrangement of inwardly facing signal member protrusions 66, the plunger rod 41 could comprise the feedback structure. For example, the plunger rod 41 could comprise a spatial sequence of indentations and/or protrusions as the feedback structure. The signal member would then comprise inwardly facing protrusions as the engaging structure, for example only at one axial position, for example near the proximal end of the arms.

In addition or as an alternative, the feedback structure may be different from comprising a spatial sequence of protrusions as illustrated in the drawings. For example, the feedback structure could comprise a spatial sequence of indentations, or the signal member (and/or the plunger rod) could generally have an undulated structure whereby the feedback structure has both, indentations and protrusions.

Further, not all of the elements described in the present text are necessarily separate parts. For example, the container housing and the housing sleeve and/or the housing sleeve and the distal end cap member could be integrated as one single part. Also, the spring guide could be integral with the signal member or with the distal end cap member (and protrude through a hole in the transversal bridge 61 into the space between the arms 62)—or could be omitted or at least have a reduced axial extension, depending on the mechanical stability of the drive spring.

Moreover, the biasing member that stores the energy for the expelling movement is not necessarily a drive spring. It could for example be a gas cartridge or other simple energy store capable of setting the drive unit into motion to expel the medicament.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the medicament delivery devices described herein include, but are not limited to, antibodies, proteins, fusion proteins, peptibodies, polypeptides, pegylated proteins, protein fragments, protein analogues, protein variants, protein precursors, and/or protein derivatives. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

Various modifications to the embodiments described are possible and will occur to those skilled in the art without departing from the invention which is defined by the following claims.

1-10. (canceled)

11. A medicament delivery device for accommodating a medicament container, the medicament delivery device comprising:
a base unit with a housing extending along a proximodistal axis;
a drive unit arranged to be subject to an expelling movement relative to the base unit into a proximal direction, to act on the medicament container for expelling a medicament therefrom; and
a signal member, the signal member extending longitudinally and being configured to interact with the drive unit and the base unit to be displaced towards distally upon completion of the expelling movement and to thereby generate a confirmation signal,
wherein one of the signal member and of the drive unit comprises a feedback structure, the feedback structure extending along the axial direction and comprising a plurality of indentations and/or protrusions, wherein the other one of the signal member and of the drive unit comprises an engaging structure, wherein the expelling movement causes the engaging structure to slide along the feedback structure and to thereby engage, one after the other, a plurality of the indentations and/or protrusions to cause a sequence of feedback signals.

12. The medicament delivery device according to claim 11, wherein the signal member has an overall U-shape with two arms extending towards proximally from a transversal bridge portion, wherein a biasing member, which biases the drive unit to be subject to the expelling movement when released, is arranged to be compressed between the transversal bridge portion and the drive unit.

13. The medicament delivery device according to claim 11, wherein the drive unit comprises a plunger rod equipped to act on a plunger or stopper of the medicament container to move it towards proximally during the expelling movement to expel the medicament from the medicament container.

14. The medicament delivery device according to claim 13, wherein the plunger rod comprises the feedback structure or the engaging structure, respectively.

15. The medicament delivery device according to claim 14, wherein the signal member, in an initial state prior to the expelling movement, at least partially accommodates the plunger rod by the two arms extending along transversal sides of the plunger rod.

16. The medicament delivery device according to claim 15, wherein the protrusions of the feedback structure comprise a plurality of inwardly facing signal member protrusions along the arms of the signal member.

17. The medicament delivery device according to claim 11, wherein the base unit a tubular portion with a cavity open towards proximally, wherein the signal member and the drive unit are, in the initial state prior to the expelling movement, at least partially accommodated in the cavity.

18. The medicament delivery device according to claim 17, wherein the signal member has the transversal bridge and the arms extending towards proximally therefrom, wherein the signal member further comprises support portions protruding towards radially-outwardly from the arms, the support portions securing the signal member from being displaced towards distally further into the cavity, wherein the drive unit has the plunger rod at least partially accommodated, in the initial state, between the arms, and wherein the arms are allowed to flex radially inwardly as soon as the plunger rod is moved, by the expelling movement, to a position proximally of the arms, whereby the support portions cease to secure the signal member from being displaced towards distally, so that the biasing member urges the signal member further into the cavity until it taps against a distal end thereof, thereby causing the confirmation signal.

19. The medicament delivery device according to claim 11, wherein the base unit comprises a distal end cap member, wherein the distal end cap member comprises a distal end wall of the medicament delivery device, and wherein the distal end cap member further comprises the tubular portion with the cavity.

20. A medicament delivery assembly, comprising the medicament delivery device according to claim 11, and further comprising the medicament container assembled with the medicament delivery device.
